(19) United States
(12) Patent Application Publication
(10) Pub. No.: US 2009/0118813 A1
(43) Pub. Date: May 7, 2009

(54) NANO-PATTERNED IMPLANT SURFACES

(76) Inventors: Torsten Scheuermann, Munich (DE); Michael Kuehling, Munich (DE); Peter Albrecht, Feldafing (DE)

Correspondence Address: FISH & RICHARDSON PC, P.O. BOX 1022, MINNEAPOLIS, MN, 55440-1022, US

(21) Appl. No.: 11/934,413

(22) Filed: Nov. 2, 2007

(51) Int. Cl. A61F 2/82; B05D 1/00

(52) U.S. Cl. 623/1.15; 427/2.24

(57) ABSTRACT

A bioerodible endoprosthesis erodes to a desirable geometry that can provide, e.g., improved mechanical properties or degradation characteristics.

### TECHNICAL FIELD

This invention relates to endoprostheses, and to methods of making the same.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, e.g., so that it can contact the walls of the lumen.

The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn from the lumen.

It is sometimes desirable for an implanted endoprosthesis to be endothelialized within a body. For example, an endothelialized endoprosthesis can decrease restenosis, which may help the passageway recover to its natural condition. The endoprosthesis can be formed of a metallic material, such as stainless steel, platinum-enhanced radiopaque stainless steel (PERSS), niobium, tantalum, titanium, or alloys thereof. It is sometimes desirable for an implanted endoprosthesis to erode over time within the passageway. For example, a fully erodible endoprosthesis does not remain as a permanent object in the body, which may help the passageway recover to its natural condition. Erodible endoprostheses can be formed from, e.g., a polymeric material, such as polylactic acid, or from a metallic material, such as magnesium, iron or an alloy thereof. The endoprosthesis can have a patterned coating, which can be formed of materials such as iridium oxide, titanium nitride, titanium oxide, niobium oxide, gold, platinum, iridium, copper, silver, poly(ethylene glycol), poly(styrene-b-isobutylene-b-styrene), or combinations thereof. The patterned coating can enhance endothelialization and decrease adhesion and proliferation of smooth muscle cells, which can decrease restenosis.

### SUMMARY

The disclosure relates to patterned endoprostheses and methods of making the endoprostheses. The pattern can facilitate selective endothelialization of the endoprosthesis surface.

In one aspect, the disclosure features a medical device including a surface defining a pattern formed of at least one repeating region including at least a first material, with two adjacent elements of the at least one repeating region spaced apart by a distance of at least one nanometer and at most about 500 nanometers.

In another aspect, the disclosure includes a method of making a medical device. The method includes forming a pattern of at least one repeating region on a surface, the at least one repeating region including a first material, with two adjacent elements of the at least one repeating region being spaced by a distance of at least one nanometer and at most about 500 nanometers.

Embodiments can include one or more of the following features.

The at least one repeating region can include a topographical pattern. The at least one repeating region can include an array of repeating elements (e.g., a topological array, an array of repeating elements, an array of repeating raised elements, an array of repeating recessed elements, and/or an array of repeating raised and recessed elements). In some embodiments, the at least one repeating region can include an electrical charge pattern. The at least one repeating region can include discontinuities in polarization and/or embedded charges. In some embodiments, the at least one repeating region can include a chemical pattern. The at least one repeating region can include discontinuities in elemental concentrations on the surface. The at least one repeating region can include a background pattern the includes a background material, such as cell-rejecting polymers and/or cell-rejecting compounds. In some embodiments, the medical device includes a surface defining one or more nano-structured patterns defined by local texture discontinuities of spatial frequencies between about 1/500 element/nm and about 1 element/nm. The one or more nano-structured patterns can include topographical patterns, chemical patterns, electrical charge patterns, background patterns, and/or combinations thereof.

The repeating elements can be raised and/or recessed. The repeating elements can have a height of at most about 20 nanometers and/or a width of at most about 50 nanometers. The two adjacent elements of the repeating region can be spaced apart by a distance of at least about one nanometer (e.g., at least about 50 nanometers).

The first material can include metal, oxide, polymer, and/or combinations thereof. For example, the first material can include iridium oxide, titanium nitride, titanium oxide, niobium oxide, gold, platinum, iridium, and/or combinations thereof. In some embodiments, the surface further includes a second material, the second material can be different from the first material. The second material can include copper, silver, poly(ethylene glycol), poly(styrene-isobutylene-styrene), and/or combinations thereof.

The medical device can be an endoprosthesis. In some embodiments, the medical device is tubular (e.g., a stent) and/or balloon extendable. The pattern can be selected wherein the pattern is selected for specific predetermined characteristic adhesion (e.g., preferential adhesion) to predetermined cells. For example, the pattern can be selected for preferential adhesion to endothelial cells. In some embodiments, the pattern is selected for controlled or minor adhesion to predetermined cells. For example, the pattern can be selected for controlled or minor adhesion to smooth muscle cells, platelets, and monocytes.

In some embodiments, forming the pattern of at least one repeating region includes coating the surface with the first material. Coating the surface with the first material can include physical vapor deposition, chemical vapor deposition, printing, spraying, and/or combinations thereof. In some embodiments, the method can further include coating the surface with a second material different from the first material. In some embodiments, the method includes generating the pattern by self-organization of the first material during coating. Forming the pattern of at least one repeating region can include structuring the pattern by masking techniques, such as lithography techniques and printing techniques. In some embodiments, forming the pattern of at least one repeating region includes plasma treating the surface. In some embodiments, the at least one repeating region can include an electrical charge pattern, which can be formed by doping and/or plasma treatment. In some embodiments, the at least one repeating region includes a chemical pattern, which can be formed by applying a coating of heterogeneous chemical element concentrations to the surface. In some embodiments, forming the pattern of the at least one repeating region includes applying a chemical coating to the surface with phase segregation occurring by a self-organizing process during solidification or temperature change.

Embodiments may have one or more of the following advantages.

The endoprosthesis may not need to be removed from a lumen after implantation. The endoprosthesis can have low thrombogenecity and high initial strength. The endoprosthesis can exhibit reduced spring back (recoil) after expansion. Lumens implanted with the endoprosthesis can exhibit reduced restenosis. The implanted endoprosthesis can have enhanced biocompatibility, for example, by promoting adhesion and proliferation of endothelial cells at the endoprosthesis surface. The implanted endoprosthesis can minimize the adhesion and proliferation of smooth muscle cells, which can decrease restenosis. In some embodiments, endothelialization can occur at a surface of an endoprosthesis, which can allow for better blood flow and/or lowered thrombogenecity. In some embodiments, enhanced endothelialization can promote faster healing, which can decrease the duration and/or dosage of anti-coagulative drugs.

Other aspects, features and advantages will be apparent from the description of the preferred embodiments thereof and from the claims.

### DESCRIPTION OF DRAWINGS

FIGS. 1A-1C are longitudinal cross-sectional views, illustrating delivery of an endoprosthesis in a collapsed state, expansion of the endoprosthesis, and the deployment of the endoprosthesis in a body lumen.

FIG. 2 is a perspective view of an endoprosthesis.

FIG. 3 is an enlarged perspective view of a portion of an endoprosthesis.

FIG. 4 is an enlarged view of a portion of an endoprosthesis.

FIG. 5 is an enlarged cross-sectional view of a portion of an endoprosthesis.

FIG. 6 is an enlarged cross-sectional view of a portion of an endoprosthesis.

FIG. 7 is an enlarged cross-sectional view of a portion of an endoprosthesis.

FIG. 8 is an enlarged cross-sectional view of a portion of an endoprosthesis.

FIG. 9 is a flow-chart of a method of making an endoprosthesis.

FIG. 10 is a perspective view of an embodiment of an endoprosthesis.

FIG. 11 is a perspective view of an embodiment of an endoprosthesis.

FIG. 12 is a scheme of a method of making an embodiment of an endoprosthesis.

FIG. 13 is a perspective view of an embodiment of an endoprosthesis.

FIG. 14 is a perspective view of an embodiment of an endoprosthesis.

### DETAILED DESCRIPTION

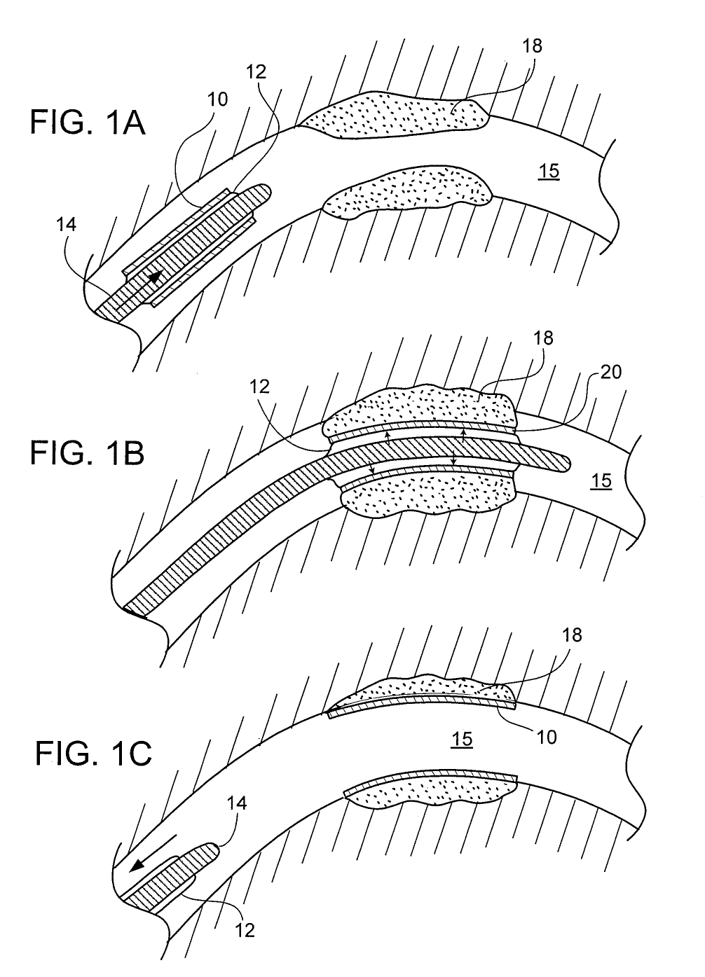

Referring to FIGS. 1A-1C, in some embodiments, during implantation of an endoprosthesis 10, the endoprosthesis is placed over a balloon 12 carried near a distal end of a catheter 14, and is directed through a lumen 15 (FIG. 1A) until the portion carrying the balloon and endoprosthesis reaches the region of an occlusion 18. The endoprosthesis is then radially expanded by inflating balloon 12 and compressed against the vessel wall with the result that occlusion 18 is compressed, and the vessel wall surrounding it undergoes a radial expansion (FIG. 1B). The pressure is then released from the balloon and the catheter is withdrawn from the vessel (FIG. 1C), leaving the endoprosthesis 10 fixed within lumen 16.

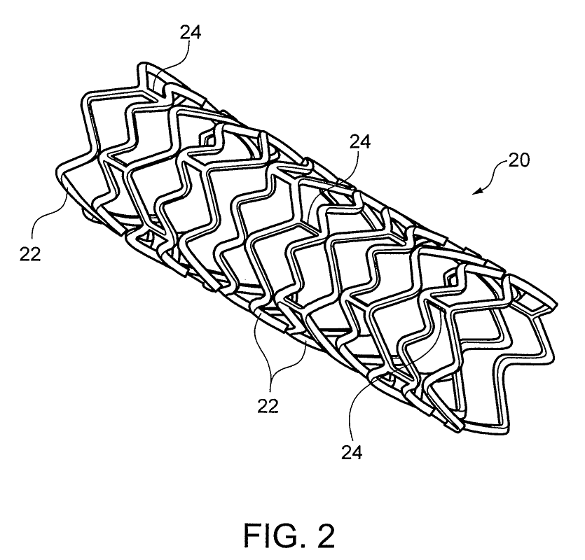

Referring to FIG. 2, an endoprosthesis 20 can include a plurality of generally circumferential struts 22 and connecting struts 24. The circumferential struts 22 can directly interconnect to one another and/or they can connect by connecting struts 24. The endoprosthesis can be delivered into a body lumen, such as a vasculature, in a reduced diameter configuration and then expanded into contact with the lumen wall to, e.g., maintain patency at the site of an occlusion. The endoprosthesis can have a patterned coating.

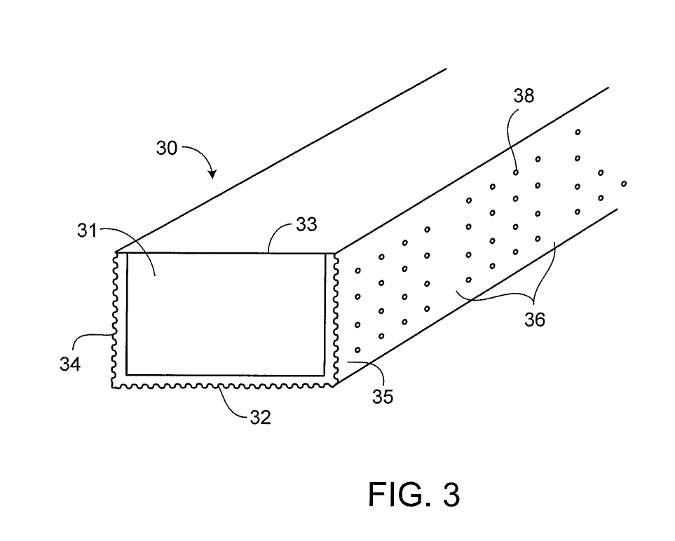

Referring to FIG. 3, an endoprosthesis having a patterned coating can selectively influence the adhesion and proliferation properties of cells. For example, an endoprosthesis having a repeating pattern can decrease the likelihood of thrombosis by selectively enhancing adhesion of certain predetermined cells, such as endothelial cells, and/or decreasing adhesion of other predetermined cells, such as smooth muscle cells, platelets, and/or monocytes. The pattern can be formed of regions having topological, chemical, or electronic features (e.g., elements). In embodiments, cells sense the surface chemistry and topography of a particular substrate to which they adhere. For example, in some embodiments, cells can react to features having a size of five nanometers or more. It is believed that cell adhesion is affected by many factors, such as differences in surface energy gradients, hydrophobicity, hydrophilicity, charge, and/or pH. These properties are affected by topological and/or chemical surface patterns. In some embodiments, a surface pattern can generate confined spaces, which can influence cell adhesion by changing local solute concentration and changing cellular wetting and protein exchange processes. In some embodiments, nanotopology influences intracellular signaling processes and cell surface receptor reorganization, which can affect cell differentiation and proliferation. Thus, a surface with a patterned coating having regions of topological, chemical, or electrical elements can help control cell proliferation, differentiation, orientation, motility, adhesion, and/or cell shape. Discussion of the effect of topographical and/or patterns on cell behavior is provided, for example, in Curtis A. et al, (1999) Biochem. Soc. Symp. 65: 15-26; in Brétagnol F. et al., (2006) Plasma Process. Polym. 3: 443-455; and in Sardella et al., (2006) Plasma Process. Polym. 3: 456-469.

In embodiments, cellular adhesion and function are generally superior on hydrophilic surfaces because of enhanced competitive binding and bioactivity of adhesion proteins such as fibronectin on hydrophilic surfaces, and/or an increased cellular ability to modify their interfacial proteins. A hydrophilic surface can have a contact angle, defined as the angle at which a liquid/vapor interface meets the solid surface, of less than or equal to 65°, while a hydrophobic surface can have a contact angle of greater than 65°. The contact angle can be measured using a contact angle goniometer. In some embodiments, a sessile drop method is used to determine the contact angle and to estimate wetting properties of a localized region on a solid surface, for example, by measuring the angle between the baseline of a drop of liquid on a surface and the tangent at the drop boundary.

Referring to FIG. 3, an enlarged perspective cross-sectional view of a strut 30, the strut is formed of a body 31 and one or more surfaces. The surface(s) can have a patterned coating having one or more regions, such that at least one region repeats at regular intervals. In some embodiments, the strut has a rectangular cross section having an adluminal surface 32, an abluminal surface 33, and side surfaces 34 and 35. All or some of the surfaces can have the same or different patterns, in any combination. For example, referring to FIG. 3, the adluminal surface 32 and the two side surfaces 34 and 35 of the strut can be covered with a pattern having regions 36 of repeating dots 38.

In some embodiments, a pattern located on the abluminal, adluminal, or the side surface of the strut can have the same topological and/or chemical patterns or different patterns. For example, an adluminal surface can contact bodily fluid more than an abluminal surface, which can contact a wall of a body passageway, and as a result, it may be more desirable to ensure rapid endothelialization of the adluminal surface compared to the abluminal surface in order to decrease thrombosis. For example, the adluminal surface can include topographical and/or chemical patterns that can enhance cell adhesion and/or proliferation to a greater degree than a pattern at abluminal surface.

In some embodiments, in addition to the patterned coating, the endoprosthesis can have a patterned background coating having a controlled or minor adhesion for certain predetermined cells, such as smooth muscle cells, platelets, and/or monocytes. In some embodiments, the background coating can be relatively hydrophobic and can decrease cellular adhesion so that cells preferentially adhere at the patterned topological and/or chemical features. The background coating can decrease the likelihood of thrombosis.

The struts can have a rectangular cross-section, a square cross-section, a circular cross-section, an ovaloid cross-section, an elliptical cross-section, a polygonal cross-section (e.g., a hexagonal, an octagonal cross-section), or an irregularly shaped cross-section. In some embodiments, a portion of the one or more strut surfaces can have a pattern. For example, one or more surfaces can have a pattern that covers at least about five percent of each surface area (e.g., at least about 10 percent, at least about 20 percent, at least about 30 percent, at least about 40 percent, at least about 50 percent, at least about 60 percent, at least about 70 percent, at least about 80 percent, or at least about 90 percent) and/or at most 100 percent of each surface area (e.g., at most about 90 percent, at most about 80 percent, at most about 70 percent, at most about 60 percent, at most about 50 percent, at most about 40 percent, at most about 30 percent, at most about 20 percent, or at most about 10 percent).

In some embodiments, the patterned coating can have one or more patterned or unpatterned regions such that the coating can be continuous or interrupted. For example, a pattern on a surface can be interrupted by multiple regions that are not patterned or have a different pattern. Each region can have an area, such that at least one dimension of the patterned region (e.g., a width, a length, and/or a diameter) is at least about 10 nm (e.g., at least about 50 nm, at least about 100 nm, at least about 500 nm, at least about one micrometer, at least about two micrometers, at least about three micrometers, at least about four micrometers, at least about five micrometers, at least about 10 micrometers). A patterned coating can selectively enhance or decrease cellular adhesion and proliferation at certain locations on an endoprosthesis.

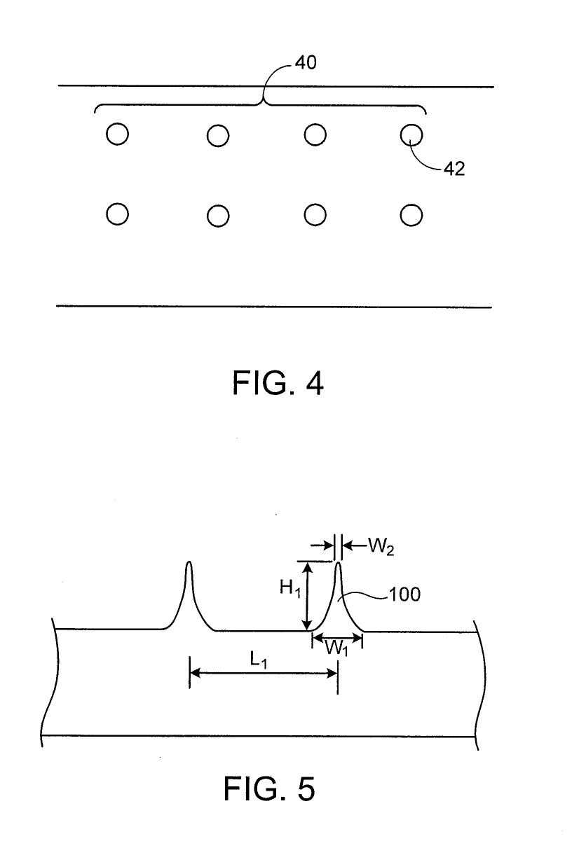

Referring to FIG. 4, the one or more regions 40 can have one or more repeating features 42 (e.g., elements). In some embodiments, the features are arranged in a square array, a hexagonal array, a brick wall array, a rectangular array, and/or a triangular array. The features can include dots, beads, spheres, columns, pillars, hills, lines, lamellae, strips, grooves, pits, circles, and/or polygonal shapes such as triangles, squares, rectangles, diamonds, and hexagons. In some embodiments, the features can be ordered or non-ordered, clustered or non-clustered, in phase or out-of-phase, parallel or non-parallel. In some embodiments, a feature is topological and differs geometrically from an endoprosthesis surface immediately surrounding the feature, such that the feature can protrude from or recess into a surface. In some embodiments, an feature is chemical and has a different composition than an endoprosthesis composition immediately surrounding the element (e.g., the matrix composition). In some embodiments, a feature is polarized and has an electric charge that is different from the area immediately surrounding each feature. The features can be distinguished from the surface by discontinuities in a surface geometry, chemical element concentration, chemical species concentration, and/or electronic polarization, or any combination thereof.

The one or more patterned regions can have at least one feature per nm (e.g., at least one feature per 10 nm, at least one feature per 15 nm, at least one feature per 25 nm, at least one feature per 50 nm, at least one feature per 75 nm, at least one feature per 100 nm, at least one feature per 200 nm, at least one feature per 300 nm, at least one feature per 400 nm) and/or at most one feature per 500 nm (e.g., at most one feature per 400 nm, at most one feature per 300 nm, at most one feature per 200 nm, at most one feature per 100 nm, at most one feature per 75 nm, at most one feature per 50 nm, at most on feature per 25 nm, at most one feature per 15 nm, or at most one feature per 10 nm).

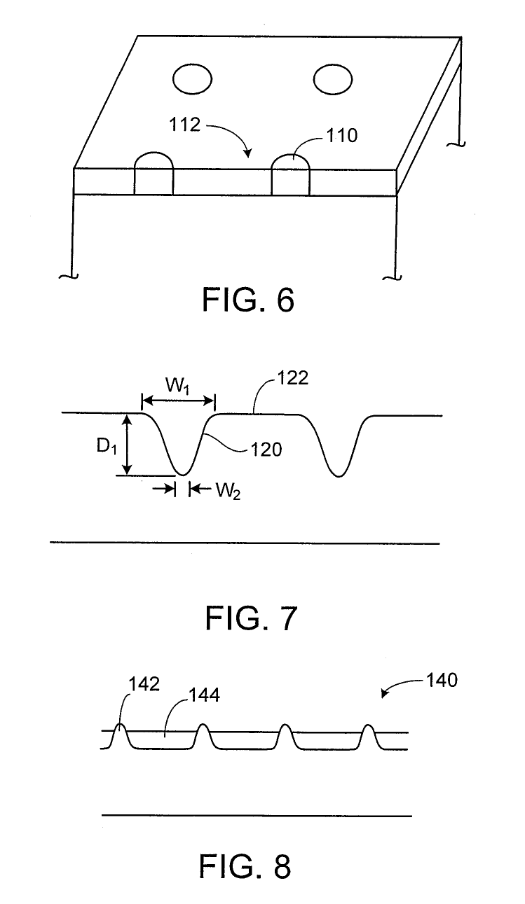

The features can have a width and a height. The width can vary or remain constant for each feature. The height can be the same or vary from one feature to another. In some embodiments, the features are at most one micrometer in width and/or height. The width and height of the features can influence cell adhesion and proliferation on an endoprosthesis surface. As an example, features having a width of about 50 nm (e.g., 25-100 nm, 25-75 nm, 25-50 nm, 10-100 nm, 10-75 nm, 10-50 nm) and/or a height of about 20 nm (e.g., 5-30 nm, 5-25 nm, 5-20 nm, 5-10 nm) can enhance endothelialization and/or decrease smooth muscle cell adhesion and proliferation. For example, referring to FIG. 5, features 100 can have a wide portion having an average width W₁ of at most about 200 nanometers (nm) (e.g., at most about 150 nm, at most about 100 nm, at most about 75 nm, at most about 50 nm, at most about 30 nm, at most about 10 nm, at most about five nm, at most about two nm, or at most about one nm). In some embodiments, features 100 can have a narrow portion having an average width W₂ of at most 50 nm (e.g., at most 40 nm, at most 30 nm, at most 20 nm, at most 10 nm, at most 5 nm, at most 3 nm, at most 2 nm, at most 1 nm). Features 100 can protrude from the surface and have a average height H₁ of at most about 200 nm (e.g., at most about 150 nm, at most about 100 nm, at most about 75 nm, at most about 50 nm, at most about 30 nm, at most about 20 nm, at most about 15 nm, at most about 10 nm, at most about five nm, at most about two nanometers, or at most about one nm). In some embodiments, such as chemical or polarized features, the features do not protrude from the surface. For example, referring to FIG. 6, features 110 can have approximately the same height as surface 112 (e.g., a chemical or electrical charge discontinuity). As another example, referring to FIG. 7, features 120 can recede into surface 122. In some embodiments, features 120 can recede into the surface by a depth D₁ of at most about 200 nm (e.g., at most about 150 nm, at most about 100 nm, at most about 75 nm, at most about 50 nm, at most about 30 nm, at most about 20 nm, at most about 15 nm, at most about 10 nm, at most about five nm, at most about two nm, or at most about one nm).

The distance separating the features can influence the adhesion and proliferation of different kinds of cells on an endoprosthesis surface. For example, an endoprosthesis having features separated by a distance of about 500 nm (e.g., from 200-500 nm, from 100-200 nm, from 100-300 nm, from 100-500 nm) can have fewer cells adhering to the endoprosthesis than an endoprosthesis having features separated by a distance of about 50 nm (e.g., from 20-50 nm, from 20-100 nm, from 50-100 nm, from 20-75 nm). Referring again to FIG. 5, features 100 can be separated by a distance L₁ of at least about one nanometer (e.g., at least 25 nanometers, at least 50 nanometers, at least 100 nanometers, at least 200 nanometers, at least 300 nanometers, at least 400 nanometers) and/or at most 500 nanometers (e.g., at most 400 nanometers, at most 300 nanometers, at most 200 nanometers, at most 100 nanometers, at most 50 nanometers, at most 25 nanometers). In some embodiments, the distance between the features can be measured by surface profilometry, where a stylus in contact with the surface of the sample can measure physical surface variations as the stylus is dragged across the surface. In some embodiments, the distance between the features can be determined using atomic force microscopy, where a topographic profile map can be interpreted by an image processing software to provide distance information between the elements.

In some embodiments, the features are formed of materials such as iridium oxide, titanium nitride, titanium oxide, niobium oxide, gold, platinum, iridium, and/or a polymer (e.g., polyethylene or polypropylene containing polymers, polylactic acid, poly(lactide-co-glycolide), poly(styrene-b-isobutylene-b-styrene), methylenebisacrylamide-containing polymers, polyethylene-co-vinyl acetate, poly n-butyl methacrylate, chondroitin sulfate, and/or gelatin). In some embodiments, the elements include a chemical moiety that enhances attachment and proliferation of certain types of cells. For example, the elements can include an amino acid sequence, such as RGD (arginine-glycine-aspartate), to enhance adhesion of cells. As another example, the elements can include carboxylic acid moieties such as a carboxylic acid-functionalized polymers or NH₂ moieties, which can enhance cell binding. Examples of carboxylic acid-functionalized polymers include polyacrylic acid, poly(maleic acid), and co- and terpolymers containing acrylic and maleic acid. Examples of NH₂-functionalized polymers include poly(allyl amine), nylons, aramids, and sodium poly(aspartate).

The features and the surrounding matrix can be formed of the same or different materials. For example, the elements and the surface can be formed of a block copolymer, which can phase separate to form elements including a first component of the block copolymer, and a background surface formed of a second component of the block copolymer. An example of a block copolymer is polystyrene-block polyethylene oxide (PS-b-PEO). The components of the block polymer can be different. Referring to FIG. 8, in some embodiments, the surface of an endoprosthesis 140 includes features 142 and a background coating 144. Background coating 144 can include a material that resists cell adhesion. As an example, background coating 144 can be formed of copper, silver, polyethylene glycol, poly(styrene-b-isobutylene-b-styrene), and/or combinations thereof.

In some embodiments, the features have a different chemical element composition than the matrix composition, and/or the features can have discontinuities in chemical element concentration compared to the matrix. As an example, the features can have a higher percentage of Au than the surface surrounding the features. The difference in one or more chemical element concentrations between the compositions of the features and the surrounding matrix can each be greater than or equal to five percent (e.g., greater than or equal to 10 percent, greater than or equal to 15 percent, greater than or equal to 20 percent, greater than or equal to 30 percent, greater than or equal to 40 percent, greater than or equal to 50 percent, greater than or equal to 60 percent, greater than or equal to 70 percent, greater than or equal to 80 percent, greater than or equal to 90 percent) and/or less than or equal to 100 percent (e.g., less than or equal to 90 percent, less than or equal to 80 percent, less than or equal to 70 percent, less than or equal to 60 percent, less than or equal to 50 percent, less than or equal to 40 percent, less than or equal to 30 percent, less than or equal to 20 percent, less than or equal to 10 percent) by weight. The chemical element distribution on a surface of the endoprosthesis can be measure by, for example, energy dispersive X-ray spectroscopy (EDX), scanning tunneling microscopy (STM), atomic force microscopy (AFM), and/or electron microprobes.

In some embodiments, cell membranes have net negative charge and adhere closely to positively charged surfaces, and/or adhere only at select sites on negatively charged surfaces. To enhance selective binding of certain predetermined cell types (e.g., endothelial cells), the features can have a different electric charge than the surrounding matrix material. For example, the features can have a larger or a smaller positive or negative charge compared to the matrix material. In some embodiments, the features and the surrounding matrix material can have different polarizations. For example, the features can have a net positive polarization, while the surrounding material can have a net negative polarization. The surface charge (e.g., polarization) can be generated by plasma treatment of a surface using a colloidal mask or through polymers having embedded charges. A surface charge is expressed by surface charge density in Coulomb per square meters (C/m²), and can be measured using an surface charge analyzer, or preferably with STM and/or AFM.

In some embodiments, the endoprosthesis can have pores, which can contain therapeutic agents that are slowly released over time. The pores can have an average diameter of from about 10 nm (e.g., from about 20 nm, from about 50 nm, from about 100 nm, from about 200 nm, from about 500 nm, from about 700 nm, from about 1 μm, from about 1.5 μm, from about 2 μm, from about 2.5 μm, from about 3 μm, from about 3.5 μm, from about 4 μm, from about 4.5 μm) to about 10 μm (e.g., to about 9 μm, to about 8 μm, to about 7 μm, to about 6 μm, to about 5 μm, to about 4.5 μm, to about 4 μm, to about 3 μm, to about 2.5 μm, to about 2 μm, to about 1.5 μm, to about 1 μm, to about 750 nm, to about 500 nm, to about 250 nm, to about 100 nm, to about 75 nm, to about 50 nm, to about 25 nm). The pores can have an average surface area of from about 300 nm² (e.g. from about 1,000 nm², from about 5,000 nm², from about 30,000 nm², from about 0.5 μm², from about 6 nm², from about 10 μm², from about 20 μm², from about 30 μm², from about 40 μm², from about 50 μm², from about 65 μm²) to about 350 μm² (e.g., to about 300 μm², to about 250 μm², to about 200 μm², to about 150 μm², to about 100 μm², to about 70 μm², to about 65 μm², to about 50 μm², to about 40 μm², to about 30 μm², to about 20 μm², to about 10 μm², to about 6 μm², to about 0.5 μm², to about 30,000 nm², to about 5,000 nm², to about 1000 nm²). The pores can also be expressed by average volume. In some embodiments, the pores can be from about 500 nm³ (e.g., from about 0.00005 μm³, from about 0.0005 μm³, from about 0.005 μm³, from about 0.05 μm³, from about 0.5 μm³, from about 1 μm³, from about 5 μm³, from about 35 μm³, from about 50 μm³) to about 550 μm³ (e.g., to about 450 μm³, to about 300 μm³, to about 200 μm³, to about 100 μm³, to about 75 μm³, to about 40 μm³, to about 10 μm³, to about 5 μm³, to about 1 μm³, to about 0.5 μm³, to about 0.05 μm³, to about 0.005 μm³, to about 0.00005 μm³).

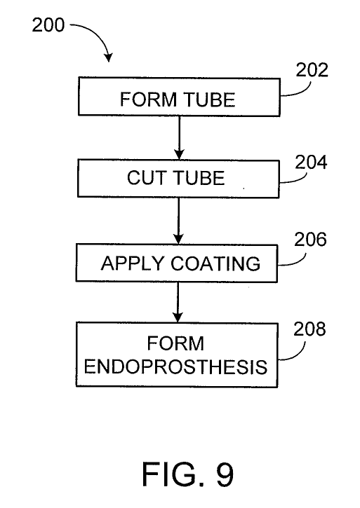

Referring to FIG. 9, a method 200 of making an endoprosthesis as described herein is shown. Method 200 includes forming a tube (step 202), forming a pre-endoprosthesis from the tube (step 204), and applying one or more patterns and/or coatings to the pre-endoprosthesis (step 206) to form an endoprosthesis. In some embodiments, one or more patterns and/or coatings are applied to the tube, and the tube is subsequently formed into an endoprosthesis.

The tube can be formed (step 202) by manufacturing a tubular member including (e.g., formed of) one or more materials capable of supporting a bodily lumen. For example, a mass of material can be machined into a rod that is subsequently drilled to form the tubular member. As another example, a sheet of material can be rolled to form a tubular member with overlapping portions, or opposing end portions of the rolled sheet can be joined (e.g., welded) together to form a tubular member. A material can also be extruded to form a tubular member. In certain embodiments, a tube can be made by thermal spraying, powder metallurgy, thixomolding, die casting, gravity casting, and/or forging. The material can be a substantially pure metallic element, an alloy, or a composite. Examples of metallic elements include iron, niobium, titanium, tantalum, magnesium, zinc, and alloys thereof. Examples of alloys include stainless steel such as platinum enhanced radiopaque stainless steel (PERSS), iron alloys having, by weight, 88-99.8% iron, 0.1-7% chromium, 0-3.5% nickel, and less than 5% of other elements (e.g., magnesium and/or zinc); or 90-96% iron, 3-6% chromium and 0-3% nickel plus 0-5% other metals. Other examples of alloys include magnesium alloys, such as, by weight, 50-98% magnesium, 0-40% lithium, 0-5% iron and less than 5% other metals or rare earths; or 79-97% magnesium, 2-5% aluminum, 0-12% lithium and 1-4% rare earths (such as cerium, lanthanum, neodymium and/or praseodymium); or 85-91% magnesium, 6-12% lithium, 2% aluminum and 1% rare earths; or 86-97% magnesium, 0-8% lithium, 2%-4% aluminum and 1-2% rare earths; or 8.5-9.5% aluminum, 0.15%-0.4% manganese, 0.45-0.9% zinc and the remainder magnesium; or 4.5-5.3% aluminum, 0.28%-0.5% manganese and the remainder magnesium; or 55-65% magnesium, 30-40% lithium and 0-5% other metals and/or rare earths. Magnesium alloys are also available under the names AZ91D, AM50A, and AE42. Other erodible materials are described in Bolz, U.S. Pat. No. 6,287,332 (e.g., zinc-titanium alloy and sodium-magnesium alloys); Heublein, U.S. Patent Application 2002000406; and Park, Science and Technology of Advanced Materials, 2, 73-78 (2001), all of which are hereby incorporated by reference herein in their entirety. In particular, Park describes Mg—X—Ca alloys, e.g., Mg—Al—Si—Ca, Mg—Zn—Ca alloys. Other suitable alloys include strontium. As an example, strontium can be a component in a magnesium alloy. The tube can include more than one material, such as different materials physically mixed together, multiple layers of different materials, and/or multiple sections of different materials along a direction (e.g., length) of the tube. An example of a composite is as a mixture of a magnesium alloy in a polymer, in which two or more distinct substances (e.g., metals, ceramics, glasses, and/or polymers) are intimately combined to form a complex material. In some embodiments, one or more materials are bioerodible.

Referring again to FIG. 9, after the tube is formed, the tube is converted into a pre-endoprosthesis (step 204). In some embodiments, selected portions of the tube can be removed to form circular and connecting struts (e.g., 6, 8) by laser cutting, as described in U.S. Pat. No. 5,780,807, hereby incorporated herein by reference in its entirety. Other methods of removing portions of the tube can be used, such as mechanical machining (e.g., micro-machining, grit blasting or honing), electrical discharge machining (EDM), and photoetching (e.g., acid photoetching). The pre-endoprosthesis can be etched and/or electropolished to provide a selected finish. In certain embodiments, such as jelly-roll type endoprostheses, step 204 is maybe omitted.

Prior to applying the patterned coating, selected surfaces (e.g., interior surface) or portions (e.g., portion between the end portions of the endoprosthesis) of the pre-endoprosthesis can be masked so that the patterned coating will not be applied to the masked surfaces or portions. In some embodiments, prior to applying the patterned coating, pores can be formed on the pre-endoprosthesis (e.g., by micro-arc surface modification, sol-gel templating processes, near net shape alloy processing technology such as powder injection molding, adding foaming structures into a melt or liquid metal, melting a powder compact containing a gas evolving element or a space holder material, incorporating a removable scaffold (e.g., polyurethane) in a metal powder/slurry prior to sintering, sintering hollow spheres, sintering fibers, combustion synthesis, powder metallurgy, bonded fiber arrays, wire mesh constructions, vapor deposition, three-dimensional printing, and/or electrical discharge compaction). In some embodiments, pores can be formed by incorporating embedded microparticles and/or compounds (e.g., a salt) within a pre-endoprosthesis (e.g., a polymerizable monomer, a polymer, a metal alloy), and removing (e.g., dissolving, leaching, burning) the microparticles and/or compounds to form pores at locations where the microparticles and/or compounds were embedded. Removable (e.g., dissolvable) microparticles can be purchased, for example, from MicroParticles GmbH. In some embodiments, pores are formed by using a gas as a porogen, bonding fibers, and/or phase separation in materials such as polymers, metals, or metal alloys.

Next, the patterned coating(s) is applied to the pre-endoprosthesis (step 206) to form an endoprosthesis. A topographical patterned coating can be formed on the endoprosthesis surface by a variety of processes, such as plasma treatment, plasma-enhanced chemical vapor deposition, and plasma etching processes. A plasma process can occur prior to applying a mask, or after. In some embodiments, a physical mask (e.g., a polymer or metal sheet with micro- or nanometer sized openings) is used in conjunction with plasma processes to provide micro-patterned surfaces. For example, plasma patterning can occur through TEM grids, and/or through nanocolloidal masks to obtain micro- and nanosized elements. In some embodiments, different composition and properties can be conferred to a surface using different plasma processes, for example, plasma deposition can deposit coating with cell adhesive-cell repulsive, acidic-basic, hydrophobic-hydrophilic properties on an endoprosthesis surface. In some embodiments, plasma deposited films are more stable and can be deposited on a wide range of substrates. The films can also have a variety of chemical functionalities, and have increased density and/or coverage. In some embodiments, plasma processes can produce non-specific cell-adhesive surfaces, for example, surfaces can contain COOH, or NH₂ groups. In certain embodiments, COOH groups can be plasma deposited from poly(acrylic acid), and NH₂ functionalized coating can be formed by grafting nitrogen containing groups onto polymers with RF glow discharges with a NH₃ feed, or using NH₂ functionalized polymers, such as poly(allylamine). Plasma deposition can also form cell-repulsive surfaces, which can be generated by plasma-depositing poly(ethylene oxide).

In some embodiments, a colloidal lithography technique can be coupled with plasma processes to generate a surface with repeating topographical elements/elements, for example, conical shaped elements. For example, a poly(acrylic acid) film can be deposited onto a substrate via plasma enhanced chemical vapor deposition of acrylic acid vapor using a capacitively coupled plasma reactor. A hexagonally assembled monolayer of colloidal particles can then be deposited onto the polymer film by spin-coating the film with a solution of the particles. Oxygen plasma etching can be carried out in a high density plasma source to generate a hexagonal topological pattern with raised poly(acrylic acid) nanostructures. In some embodiments, plasma etching through a mask can form an array of recessed elements. In other embodiments, a cell-repulsive poly(ethylene oxide) film can be deposited via plasma polymerization, and ultrasound washing can remove any remaining colloidal particle masks. Colloidal lithography can form features having a maximum dimension of less than 50 nm (e.g., less than 40 nm, less than 30 nm, less than 20 nm, less than 10 nm, less than 5). The dimension of the features can vary depending on the size of the colloidal particles, where smaller particles can afford smaller features, and larger particles can afford larger features. Examples of colloidal particles include Au, Ag, Cr, or polymer (e.g., polystyrene) spheres. Discussion of combined colloidal lithography and plasma sputtering or etching methods is provided, for example in Sardella et al., (2006) Plasma Process. Polym. 3: 456-469; Valsesia et al, (2004) Nano Lett., 4: 1047-1050; and Brétagnol et al., 2006 Plasma Process. Polym. 3: 443-455.

As an example, in some embodiments, polystyrene-block polyethylene oxide (PS-b-PEO) is used as a micelle-forming block copolymer, and Au is used for small particles to be generated inside the micelles. PS-b-PEO can self-assemble to form micelles in a non-polar solvent (e.g., toluene). When LiAuCl₄ is added to a solution of PS-b-PEO, the salt can be slowly solubilized as the Li+ ions form a complex with the polyethylene oxide units of the block copolymer forming the micellar structures. The tetrachloroaurate ions can be bound as counterions within the core of the micelle. Solubilization can be facilitated by means of ultrasound. Typically, up to 0.3 equivalents of LiAuCl₄ can be bound per ethylene oxide. Using larger quantities of LiAuCl₄ can lead to precipitation of unbound LiAuCl₄. Complex formation of the polyethylene oxide block with LiAuCl₄ can considerably enhance the stability of the PEO micelles. When deposited on a substrate, the PS-b-PEO films can be monolayers and can have a thickness of less than or equal to 100 nm, depending on the polymer length of the micelles. The PS-b-PEO can be removed through heating or plasma treatment, leaving the Au colloids on the surface of a substrate having inter-colloid distances correlating to the micelle lengths of the PEO.

In some embodiments, in addition or as an alternative to plasma deposition, cell-adhesive or repulsive polymer films can be deposited by physical adsorption, radiation, chemical cross-linking, self-assembly, spin coating, chemisorption, and/or treating with ion beams. In some embodiments, the coating can be a composite, such as a silver-containing coating which can be used to reduce bacteria colonization. A composite coating can be obtained by various methods, such as sol-gel, high temperature glass fusion, and/or ion exchange methods. In some embodiments, an organic matrix is deposited from the fragments of an organic, volatile monomer, and metal (or ceramic, or polymer) particles are co-deposited from a sputtering (or etching, evaporation or PE-CVD process. Discussion of composite film coating processes is provided, for example, in Sandella et al., supra.

In some embodiments, block copolymer micelle nanolithography is used to make a coating of hexagonally close-packed array of gold nanodots. The gold nanodots can be coated with cyclic RGDFK peptide linked to the nanodot via a spacer (e.g., aminohexanoic acid linked to mercaptopropionic acid), and the polymer can be polystyrene-block-poly(2-vinylpyridine). In some embodiments, the diameter of dots is 20 nm or less (e.g., 10 nm or less, 8 nanometers or less). The spacing between the nanodots can be controlled by selecting an appropriate segment molecular weight and the composition for the block copolymer. In some embodiments, spacing between the nanodots can be less than 500 nm (e.g., less than 400 nm, less than 300 nm, less than 200 nm, less than 100 nm, less than 500 nm). Discussion of methods of making patterned nanodots is provided, for example, in Arnold et al., (2004) Chem Phys Chem 5: 383-388.

In some embodiments, the patterned coating and/or background coating can be made by ink-jet printing, spraying, physical vapor deposition, chemical vapor deposition, stretching, photolithography, soft lithography, dip-pen lithography, nano-fountain-pen lithography, colloidal lithograph, hot-embossing, electrolytic etching, and/or extrusion. For example, when a patterned coating is made by lithography, the surface to be patterned can be coated with a thin layer of photosensitive polymer such as a photoresist, which is then exposed to the appropriate illumination through a patterned mask, and subsequently chemically developed or irradiated with an electron beam to reveal the underlying substrate and features. In some embodiments, the exposed patterned substrate can react with a chemical linker, such as an amino-functionalized thiol, which can react with glutaraldehyde and/or proteins to enhance the biocompatibility of the endoprosthesis. In some embodiments, the patterned endoprosthesis can be functionalized with attachment factors such as vitronectin, fibronectin, and/or laminin to create regions that can influence cellular adhesion, growth, and survival. Discussion of methods of generating patterned coatings is provided, for example, in Curtis A. et al., (1999) Biochem. Soc. Symp. 65: 15-26. Discussion of methods of functionalizing substrates is provided, for example, in Clark, Immobilized Biomolecules in Analysis—A Practical Approach. Eds: Tony Cass and Frances S. Ligler, Oxford University Press. 1998. pages 95-111.

In some embodiments, self-organizing systems such as polymer demixing, self-assembling particles and monolayers, self-assembling polymers can form repeating features and/or background coating. The features can have a maximum dimension of 100 nm or less (e.g., 80 nm or less, 60 nm or less, 40 nm or less, 20 nm or less, 10 nm or less, 5 nm or less). For example, the patterned coating can be made by self assembly of block copolymers, such that repeating areas of a segment of the block copolymer can be achieved by phase separation (e.g., during solidification and/or temperature change). As another example, the patterned coating can be made by polymer demixing, which can form structures such as islands of polymers. For example, a solution of polystyrene-blend-polybromostyrene and polystyrene-blend-poly(n-butyl methacrylate) can result in different topographies depending on the polymer concentration and the speed with which a solvent is removed from the mixture. The mixture can form islands having a height of less than 200 nm (e.g., less than 100 nm) with mean diameter of less than 1000 nm (e.g., less than 500 nm, less than 400 nm, less than 300 nm, less than 200 nm, less than 100 nm) at pressures of 1 psi. At increased pressures, ribbons of polymers having shallower features and decreased separation between the structures can form. At increasing polymer concentrations, structures having an increased height (e.g., from 200-400 nm, from 200-300 nm, from 250-400 nm, from 250-300 nm) can result. Discussion of polymer demixing is provided, for example, in Gadegaard et al., 2004 Adv. Mater. 16(20): 1857-1860.

In some embodiments, the endoprosthesis can have an electronic pattern. The electronic pattern can be formed by doping an endoprosthesis, for example, by implanting doping elements using ion accelerators (ion beam) and a colloidal lithographic mask.

In some embodiments, the endoprosthesis can have discontinuities in elemental concentrations that form a pattern. Elemental discontinuities can be formed, for example, by ion implantation, reactive physical vapor deposition (PVD) and chemical vapor deposition (CVD) processes.

Examples of suitable patterned coating materials include compounds such as gold, platinum, iridium, titanium, silicon, carbon, silica, titanium dioxide, lithium niobate, iridium oxide, titanium nitride, niobium oxide, and/or silicon nitride; polymers such as poly(methylmethacrylate), polydioxanone, polystyrene, polylactide, polyglycolides, cellulose acetate, polyurethane, silicone, epoxy, nylon, cellulose acetate, polyimide; biomolecules such as collagen, and/or fibrin. Examples of suitable materials for cell-rejecting background coatings include copper, silver, poly(ethylene oxide), poly(ethylene glycol), and/or poly(styrene-isobutylene styrene). Discussion of topologically or chemically patterned coatings is provided, for example, in Curtis et al., (1997) Biomaterials. 18:1573-1583 and Curtis et al., (1997) Biochem. Soc. Symp. 65: 15-26.

Further examples of patterned coating and/or background materials include a polymers, ceramic materials, oxides, carbides, halides, metals, metallic alloys, and/or a metal-containing polymers. For example, suitable polymers include bioerodible polymers as polylactic acid (PLA), polylactic glycolic acid (PLGA), polyanhydrides (e.g., poly(ester anhydride)s, fatty acid-based polyanhydride, amino acid-based polyanhydride), polyesters, polyester-polyanhydride blends, polycarbonate-polyanhydride blends, and/or combinations thereof. Suitable ceramic materials include, for example, iridium oxide. Suitable oxides include magnesium oxide, titanium oxide, and/or aluminum oxide. Suitable nitrides include magnesium nitride, titanium nitride, titanium oxynitride, iron nitride, and/or silicon nitride. Suitable carbides include iron carbide and silicon nitride. Suitable halides include magnesium fluoride. Suitable metals and/or a metallic alloys include stainless steel, titanium, niobium, a radiopaque metal such as gold, platinum, iridium, and alloys thereof; an alloy such as bioerodible magnesium alloys and iron alloys as previously described having adjusted compositions so that erosion occurs at a different rate than the bioerodible body. Suitable inert or dissolvable polymers including metals (e.g., Fe, Au, Pt) or metal compounds such as organometallic complexes. PVD and PLD deposition techniques are described in U.S. patent application Ser. No. 11/752,735 and U.S. patent application Ser. No. 11/752,772.

In some embodiments, the endoprosthesis includes patterned and/or unpatterned coatings. Depending on the coating material, one or more material can be dissolved in a solvent and applied to the pre-endoprosthesis, and/or two or more different materials can be blended together in the form of, for example, a composite such as a metal matrix composite (e.g., in a manner that one material is embedded or encapsulated in a remaining material) and applied to the pre-endoprosthesis. In some embodiments, an endoprosthesis coating is generated by physical or plasma vapor deposition, thermal metal spraying, dip coating, electrostatic spraying, conventional air atomization spraying, ion implantation (e.g., by plasma immersion ion implantation, by laser-driven ion implantation), electrochemical deposition, oxidation (e.g., anodizations), chemical grafting, interlayer transitional coatings to bond multiple layers, and/or metallurgical augmentation (e.g., peening, localized metallurgical treatments). In some embodiments, pores are generated in the coating, e.g., by powder injection molding sol-gel templating processes, near net shape alloy processing technology such as powder injection molding, micro-arc surface modification, sol-gel templating processes, adding foaming structures into a melt or liquid metal, melting a powder compact containing a gas evolving element or a space holder material, incorporating a removable scaffold (e.g., polyurethane) in a metal powder/slurry prior to sintering, sintering hollow spheres, sintering fibers, combustion synthesis, powder metallurgy, bonded fiber arrays, wire mesh constructions, vapor deposition, three-dimensional printing, and/or electrical discharge compaction). In some embodiments, pores can be formed by incorporating embedded microparticles and/or compounds (e.g., a salt) within the coating (e.g., a polymerizable monomer, a polymer, a metal alloy), forming the coating, and removing (e.g., dissolving, leaching, burning) the microparticles and/or compounds to form pores at locations where the microparticles and/or compounds were embedded. Removable (e.g., dissolvable) microparticles can be purchased, for example, from MicroParticles GmbH. In some embodiments, pores are formed by using a gas as a porogen, bonding fibers, and/or phase separation in materials such as polymers, metals, or metal alloys.

In some embodiments, a medicament is incorporated into a coating on an endoprosthesis. For example, a medicament can be adsorbed onto a coating on an endoprosthesis. A medicament can be encapsulated in a bioerodible material and embedded in a coating on an endoprosthesis. As another example, a medicament can be dissolved in a polymer solution and coated onto an endoprosthesis. Incorporation of a medicament is described in U.S. Ser. No. 10/958,435 filed Oct. 5, 2004, hereby incorporated herein by reference.

In some embodiments, an endoprosthesis can have greater than one type of patterned coating located at the same or different locations on the endoprosthesis. As an example, an endoprosthesis can have a patterned and/or unpatterned polymer coating superimposed upon a stainless steel coating. As another example, an endoprosthesis can have a patterned and/or unpatterned polymer and metal composite coating on an exterior surface, and a patterned and/or unpatterned polymer coating on an interior surface of a strut. In certain embodiments, a patterned coating can be applied to a pre-endoprosthesis in one layer, or in multiple layers (e.g., at least two layers, at least three layers, at least four layers, at least five layers) in order, for example, to provide greater control over the thickness of a patterned coating. As an example, the intermediate portion of an endoprosthesis can have a smaller thickness of a patterned coating than the end portions of the endoprosthesis, which can contain a patterned coating having a greater thickness. The patterned and/or unpatterned coating can be applied the same way or in different ways. For example, a first, innermost coating can be plasma-deposited on the pre-endoprosthesis, and a second, outer coating can include a polymer that is dip-coated onto the first layer.

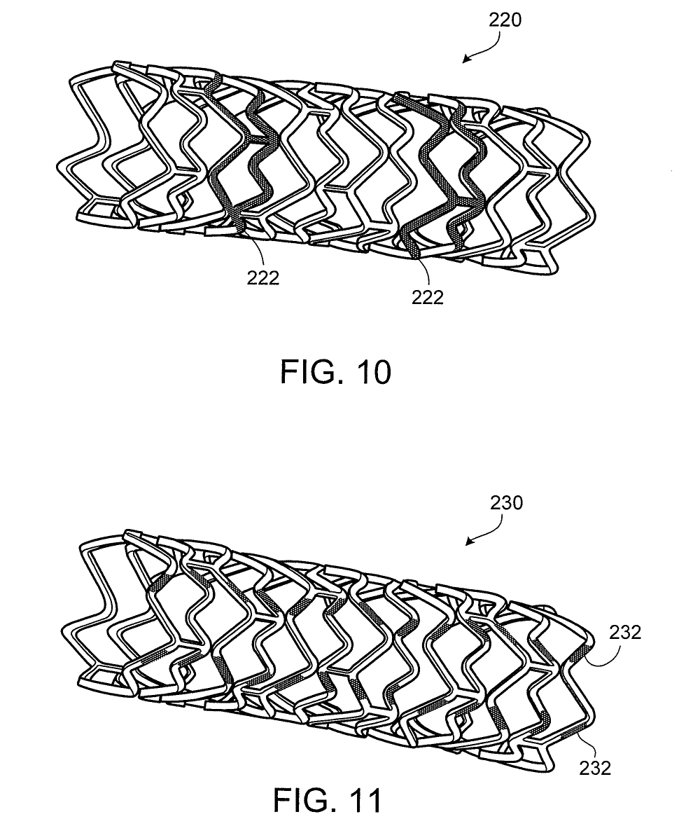

In some embodiments, a coating partially coats one or more portions of an endoprosthesis. Referring to FIG. 10, as an example, an endoprosthesis 220 can have a band(s) 222 of the same or different coatings about the circumference of the endoprosthesis. As shown in FIG. 11, as an example, an endoprosthesis 230 can have a strip(s) 232 of the same or different coatings along the length of the endoprosthesis. Bands and strips can be coated onto the endoprosthesis by selectively masking certain areas of the endoprosthesis. Bands and strips of patterned coating can have pore/patterns, and/or have different thicknesses as discussed above.

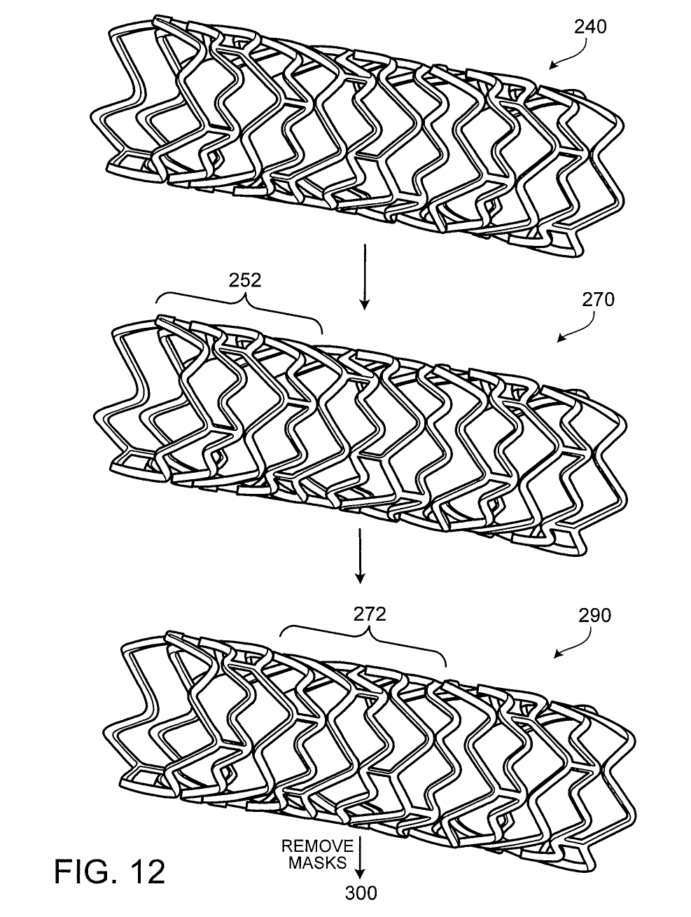

Referring now to FIG. 12, an endoprosthesis 300 having different patterned coatings along its length can be produced. A metallic pre-endoprosthesis 240 has all portions of the pre-endoprosthesis having a first coating. Next, a portion 252 of the pre-endoprosthesis is masked (e.g., with a protective polymeric coating such as a styrene-isoprene-butadiene-styrene (SIBS) polymer), which protects the masked portion from further layer coating, and the remaining section is coated with a second coating to make a pre-endoprosthesis 270. Finally, a second portion 272 of the pre-endoprosthesis is masked, and the remaining portion is further coated with a third coating to make pre-endoprosthesis 290. The protective coatings can be removed, e.g., by rinsing in a solvent such as toluene, to complete the production of endoprosthesis. An endoprosthesis having tapered thicknesses can be produced by masking the interior and/or outer portions with a movable sleeve and longitudinally moving the sleeve and/or the endoprosthesis relative to each other during coating.

In some embodiments, the patterned and/or unpatterned coating can be applied to a bioerodible tube prior to forming the bioerodible tube into an endoprosthesis. As a result, the endoprosthesis can have its exterior and interior surfaces coated with the coating, and the side surfaces of the endoprosthesis can be free of the coating. Prior to applying the patterned coating, the interior surface or the exterior surface of the bioerodible tube can be masked to apply the patterned coating to only selected portion(s) of the tube.

As another example, while the endoprosthesis can have both exterior and interior surfaces coated with a desired coating, in other embodiments, one or more segments of an endoprosthesis have only the exterior surfaces or the interior surfaces coated with a coating.

Exterior surfaces of a pre-endoprosthesis can be coated with a coating material, e.g., by placing a mandrel, a pin or a sleeve that is sized to mate with the selected inner surface(s) of the pre-endoprosthesis so that during coating, the coating material is effectively blocked from entering interior surface of the pre-endoprosthesis. Such an endoprosthesis, after implantation, may have a cross-section that has only two materials: an exterior surface that is coated with the coating material, and an interior surface that has not been coated. Interior surfaces of a pre-endoprosthesis can be coated with a desired coating material, e.g., by placing a polymeric coating on selected outer surface(s) of the pre-endoprosthesis so that during coating the composition can coat only the interior surface(s) and is prevented from coating the exterior surfaces. Alternatively, exterior surfaces can be protected by placing the pre-endoprosthesis in a tight-fitting tube, e.g., a heat shrink tube, to cover the exterior surfaces. In some embodiments, photo-lithography and/or stereo-lithography can be used to mask surfaces of a pre-endoprosthesis to prevent coating of a composition. In use, the endoprostheses can be used, e.g., delivered and expanded, using a catheter delivery system, such as a balloon catheter system. Catheter systems are described in, for example, Wang U.S. Pat. No. 5,195,969, Hamlin U.S. Pat. No. 5,270,086, and Raeder-Devens, U.S. Pat. No. 6,726,712.

Endoprosthesis and endoprosthesis delivery are also exemplified by the Radius® or Symbiot® systems, available from Boston Scientific Scimed, Maple Grove, Minn. The endoprostheses described herein can be of a desired shape and size (e.g., coronary stents, aortic stents, peripheral vascular stents, gastrointestinal stents, urology stents, and neurology stents). Depending on the application, the stent can have a diameter of between, for example, 1 mm to 46 mm. In certain embodiments, a coronary stent can have an expanded diameter of from about 2 mm to about 6 mm. In some embodiments, a peripheral stent can have an expanded diameter of from about 5 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm.

While a number of embodiments have been described, the invention is not so limited.

The endoprostheses described herein can be a part of a stent, a covered stent or a stent-graft. For example, an endoprosthesis can include and/or be attached to a biocompatible, non-porous or semi-porous polymer matrix made of polytetrafluoroethylene (PTFE), expanded PTFE, polyethylene, urethane, or polypropylene.

The endoprostheses described herein can include non-metallic structural portions, e.g., polymeric portions. The polymeric portions can be erodible. The polymeric portions can be formed from a polymeric alloy. Polymeric stents have been described in U.S. patent application Ser. No. 10/683,314, filed Oct. 10, 2003; and U.S. patent application Ser. No. 10/958,435, filed Oct. 5, 2004, the entire contents of each is hereby incorporated by reference herein.

The endoprostheses can include a releasable therapeutic agent, drug, or a pharmaceutically active compound, such as described in U.S. Pat. No. 5,674,242, U.S. Ser. No. 09/895,415, filed Jul. 2, 2001, U.S. Ser. No. 11/111,509, filed Apr. 21, 2005, and U.S. Ser. No. 10/232,265, filed Aug. 30, 2002. The therapeutic agents, drugs, or pharmaceutically active compounds can include, for example, anti-thrombogenic agents, antioxidants, anti-inflammatory agents, anesthetic agents, anti-coagulants, and antibiotics. The therapeutic agent, drug, or a pharmaceutically active compound can be dispersed in a polymeric coating carried by the endoprosthesis. The polymeric coating can include more than a single layer. For example, the coating can include two layers, three layers or more layers, e.g., five layers. The therapeutic agent can be a genetic therapeutic agent, a non-genetic therapeutic agent, or cells. Therapeutic agents can be used singularly, or in combination. Therapeutic agents can be, for example, nonionic, or they may be anionic and/or cationic in nature. An example of a therapeutic agent is one that inhibits restenosis, such as paclitaxel. The therapeutic agent can also be used, e.g., to treat and/or inhibit pain, encrustation of the endoprosthesis or sclerosing or necrosing of a treated lumen. Any of the above coatings and/or polymeric portions can be dyed or rendered radio-opaque.

The endoprostheses described herein can be configured for non-vascular lumens. For example, it can be configured for use in the esophagus or the prostate. Other lumens include biliary lumens, hepatic lumens, pancreatic lumens, uretheral lumens and ureteral lumens.

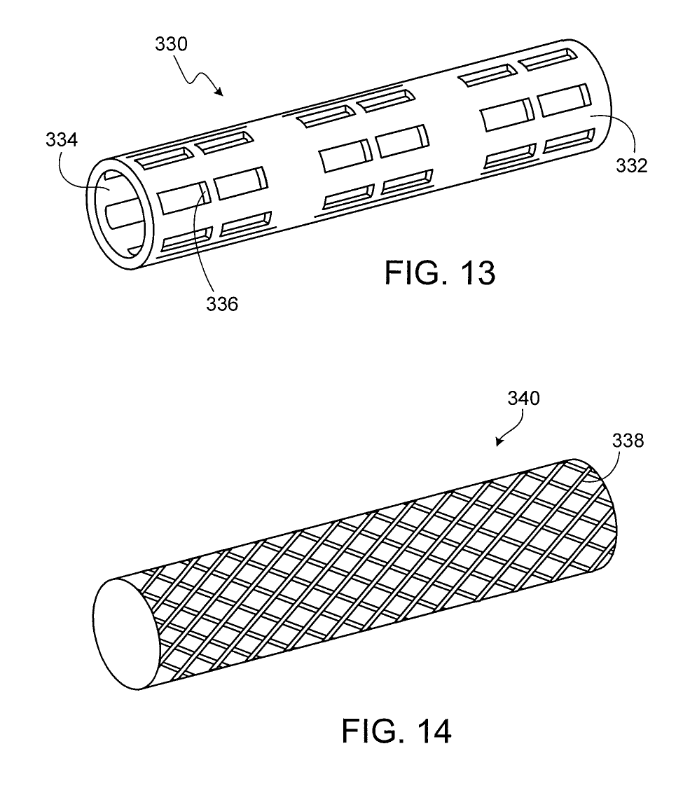

Other configurations of endoprosthesis are also possible. Referring to FIG. 13, an endoprosthesis 330 can have a tubular body with slots removed from the tubular body, an patterned and/or unpatterned coating can be coated onto an exterior surface 332, an interior surface 334, or any of the side surfaces 336 of the endoprosthesis. Referring to FIG. 14, an endoprosthesis 340 can have a braided or woven tubular body made of intertwining filaments 338. The endoprosthesis can be coated with a patterned and/or unpatterned coating on the exterior or the interior of the tubular body. In some embodiments, a braided endoprosthesis can include filaments having patterned and/or unpatterned coatings.

All references, such as patent applications, publications, and patents, referred to herein are incorporated by reference in their entirety.

Other embodiments are within the claims.

What is claimed is:

1. A medical device, comprising:
a surface defining a pattern formed of at least one repeating region comprising at least a first material, with two adjacent elements of the at least one repeating region spaced apart by a distance of at least one nanometer and at most about 500 nanometers.

2. The medical device of claim 1, wherein the at least one repeating region comprises a topographical pattern.

3. The medical device of claim 2, wherein the at least one repeating region comprises an array of repeating elements.

4. The medical device of claim 3, wherein the repeating elements are raised, recessed, or combinations thereof.

5. The medical device of claim 1, wherein the at least one repeating region comprises an electrical charge pattern.

6. The medical device of claim 1, wherein the at least one repeating region comprises a chemical pattern.

7. The medical device of claim 1, wherein the at least one repeating region comprises a background pattern comprising a background material.

8. The medical device of claim 7, wherein the background material is selected from the group consisting of cell-rejecting polymers and cell-rejecting compounds.

9. The medical device of claim 3, wherein the repeating elements has a height of at most about 20 nanometers.

10. The medical device of claim 3, wherein the repeating elements have a width of at most about 50 nanometers.

11. The medical device of claim 1, wherein the two adjacent elements of the at least one repeating region are spaced apart by a distance of at least about 50 nanometers.

12. The medical device of claim 1, wherein the first material is selected from the group consisting of metal, oxide, polymer, and combinations thereof.

13. The medical device of claim 12, wherein the first material is selected from the group consisting of iridium oxide, titanium nitride, titanium oxide, niobium oxide, gold, platinum, iridium, and combinations thereof.

14. The medical device of claim 1, wherein the surface further comprises a second material different from the first material.

15. The medical device of claim 14, wherein the second material is selected from the group consisting of copper, silver, poly(ethylene glycol), poly(styrene-isobutylene-styrene), and combinations thereof.

16. The medical device of claim 1, wherein the device is a stent.

17. The medical device of claim 1, wherein the pattern is selected for preferential adhesion to endothelial cells.

18. The medical device of claim 1, wherein the pattern is selected for controlled or minor adhesion to smooth muscle cells, platelets and monocytes.

19. A method of making a medical device, the method comprising:
forming a pattern of at least one repeating region on a surface, the at least one repeating region comprising a first material, with two adjacent elements of the at least one repeating region being spaced by a distance of at least one nanometer and at most about 500 nanometers.

20. The method of claim 19, wherein forming the pattern of at least one repeating region comprises coating the surface with the first material.

21. The method of claim 20, wherein coating the surface with the first material comprises a method selected from the group consisting of physical vapor deposition, chemical vapor deposition, printing, spraying, and combinations thereof.

22. The method of claim 19, wherein the first material is selected from the group consisting of metal, oxide, polymer, and combinations thereof.

23. The method of claim 22, wherein the first material is selected from the group consisting of iridium oxide, titanium nitride, titanium oxide, niobium oxide, gold, platinum, iridium, and combinations thereof.

24. The method of claim 20, further comprising coating the surface with a second material different from the first material.

25. The method of claim 24, wherein the second material is selected from the group consisting of copper, silver, poly(ethylene glycol), poly(styrene-isobutylene-styrene), and combinations thereof.

26. The method of claim 19, wherein the at least one repeating region comprises a topographical array of repeating elements.

27. The method of claim 20, further comprising generating the pattern by self-organization of the first material during coating.

28. The method of claim 19, wherein forming the pattern of at least one repeating region comprises structuring the pattern by masking techniques selected from the group consisting of lithography techniques and printing techniques.

29. The method of claim 19, wherein forming the pattern of at least one repeating region comprises plasma treating the surface.

30. The method of claim 19, wherein the two adjacent elements of the at least one repeating region are spaced apart by a distance of at least about 50 nanometers.
